(19) United States
(12) Patent Application Publication
(10) Pub. No.: US 2014/0178978 A1
(43) Pub. Date: Jun. 26, 2014

(54) DISTANCE-BASED QUANTITATIVE ANALYSIS USING A CAPILLARITY-BASED ANALYTICAL DEVICE

(71) Applicant: COLORADO STATE UNIVERSITY RESEARCH FOUNDATION, Fort Collins, CO (US)

(72) Inventors: David M. Cate, Fort Collins, CO (US); Josephine C. Cunningham, Austin, TX (US); Charles S. Henry, Fort Collins, CO (US); John Volckens, Fort Collins, CO (US)

(21) Appl. No.: 14/049,155

(22) Filed: Oct. 8, 2013

Related U.S. Application Data
(60) Provisional application No. 61/711,064, filed on Oct. 8, 2012.

(51) Int. Cl. G01N 33/52; G01N 21/78; C12Q 1/54

(52) U.S. Cl. CPC G01N 33/521; C12Q 1/54; G01N 21/78
USPC 435/287.7; 422/402

(57) ABSTRACT

Apparatus for quantitative analytical measurements using capillarity-based analytical devices is described. Porous cellulose (i.e., common filter paper) may be used as the reagent carrier for the analyses. Hydrophobic materials may be printed onto the paper to generate paths that restrict liquid flow by capillary action to defined regions. At least one colorimetric reagents effective for reacting with a specific analyte is deposited along a capillary flow path generated in the device. Upon placing the liquid containing the analyte on one end of the path, the liquid moves along the circuit by capillary action, and the flowing analyte reacts with reagent generating color along the flow path until all of the analyte is consumed. Analyte quantification is achieved by measuring the length of the colored portion along a flow path employing a direct-reading measurement scale. Analyses are demonstrated for chemical detection modalities including: enzymatic action, metal complexation, and nanoparticle aggregation, but the present apparatus may be used for a wide range of analytes.

### RELATED CASES

The present patent application claims the benefit of Provisional Patent Application Ser. No. 61/711,064 filed on 8 Oct., 2012 entitled “Distance-Based Detection For Capillarity-Based Analytical Devices” by Charles S. Henry et al., the disclosure and teachings of which are hereby incorporated by reference herein.

### STATEMENT REGARDING FEDERAL RIGHTS

This invention was made with government support under Grant Numbers R21 OH010050 and T42 OH009229 awarded by the Centers for Disease Control. The government has certain rights in the invention.

### FIELD OF THE INVENTION

Embodiments of the present invention relate generally to paper-based analytical devices and, more particularly, to the use of capillarity-based analytical devices for quantitative analyses employing a direct-reading measurement scale.

### BACKGROUND OF THE INVENTION

Many technological advancements in the field of measurement science focus on increasing sample throughput, sample detection limit, and the speed of sample analysis. However, such technological advancements are often limited to laboratory use by trained scientists and technicians. Consequently, there is a growing need to augment powerful modern analytical tools with low-cost methods designed for use at the point-of-need.

Point-of-need measurement technologies are often simple and inexpensive, sacrificing detection limit and operating range for sensitivity, specificity, and speed. Point-of-need technologies enable fast measurements at the place of need, at minimal cost, and with minimal user training. Examples include technologies such as litmus paper or the home pregnancy test, both of which have diffused far into everyday societal contexts. Common to each of these point-of-need devices is their reliance on simple capillarity-based flow for the analytics.

Paper-based analytical devices (PADs) represent a new generation of capillarity-based analytic devices that hold great potential for application at the point-of-need. PADs were introduced in 2007 as a tool for multiplexed assays using porous cellulose (for example, common filter paper) to store reagents and the addition of water to generate flow via capillary action. Hydrophobic materials printed onto the paper define circuits that restrict flow to defined regions. To conduct chemical analyses, colorimetric reagents are added to specific zones within the paper, with analyte detection and quantification carried out by changes in color hue and/or intensity. Although simple, this detection method has limitations, including user variability when distinguishing changes in reagent hue and intensity. Consequently, even with PADs, precise and accurate quantification can require the use of peripheral technologies such as digital scanners, cameras, or other optical techniques.

### SUMMARY OF THE INVENTION

Embodiments of the present invention overcome the disadvantages and limitations of prior art by providing an apparatus for analyte quantification employing capillarity-based analytic devices without the need to differentiate color hues and intensities.

Another object of embodiments of the present invention is to provide an apparatus for analyte quantification employing capillarity-based analytic devices and using straightforward distance measurements, without the need to differentiate color hues and intensities.

To achieve the foregoing and other objects, and in accordance with the purposes of the present invention, as embodied and broadly described herein, the apparatus for paper-based quantitative analysis of an analyte dissolved in a liquid hereof, includes: an elongated substrate effective for wicking the liquid; means for confining the liquid to a defined elongated path having a first end along the substrate, forming thereby a capillary flow path into which at least one colorimetric reagent effective for reacting with a specific analyte is deposited; means for introducing a chosen portion of said liquid into the capillary flow path of the elongated path at a location in the region of the first end thereof; whereby as said liquid moves along the capillary flow path of the elongated path away from the first end by capillary action, the flowing analyte reacts with the at least one reagent such that color develops along the flow path to a distance from the location of introduction thereof where all of the analyte is reacted; and means for measuring the distance between the region of the first end of the elongated path and the location of where all of the analyte is reacted.

In another aspect of the present invention and in accordance with its objects and purposes, the apparatus for capillarity-based, quantitative analysis of an analyte dissolved in a liquid hereof, includes: an elongated substrate effective for wicking the liquid; a liquid repelling material applied to the substrate such that an elongated path is defined for confining the liquid to a defined elongated path having a first end along said substrate, forming thereby a capillary flow path into which at least one colorimetric reagent effective for reacting with the analyte, is deposited; a syringe for introducing a chosen portion of the liquid into capillary flow path of the elongated path at a location in the region of the first end thereof; whereby the liquid moves along the capillary flow path of the elongated path away from the first end by capillary action and, as the flowing analyte reacts with the at least one reagent, color develops along the flow path to a distance from the location of introduction thereof where all of the analyte is reacted; and a measurement scale printed on the substrate for measuring the distance between the region of the first end of the elongated path and the location of where all of the analyte is reacted.

In yet another aspect of the present invention and in accordance with its objects and purposes, the apparatus for capillarity-based, quantitative analysis of an analyte dissolved in a liquid hereof, includes: an elongated substrate having a top surface and a bottom surface, effective for wicking the liquid; a liquid repelling material applied to the substrate such that an elongated path is defined for confining the liquid to a defined elongated path having a first end along said substrate, forming thereby a capillary flow path into which at least one colorimetric reagent effective for reacting with the analyte, is deposited; a syringe for introducing a chosen portion of the liquid into capillary flow path of the elongated path at a location in the region of the first end thereof; whereby the liquid moves along the capillary flow path of the elongated path away from the first end by capillary action and, as the flowing analyte reacts with the at least one reagent, color develops along the flow path to a distance from the location of introduction thereof where all of the analyte is reacted; a first transparent liquid impervious layer in contact with the top surface of the substrate, and a second liquid impervious layer in contact with the bottom surface of the substrate, the first layer and the second layer forming a seal around said substrate, and wherein the first layer has an orifice therein in the region of the first end of the elongated path opening to the substrate; and a direct-reading measurement scale printed on the first liquid impervious layer for measuring the distance between the region of the first end of the elongated path and the location where all of the analyte is reacted.

Benefits and advantages of the present invention include, but are not limited to, an apparatus for capillarity-based quantitative analysis of an analyte dissolved in a liquid, using straightforward measurements along a direct-reading distance scale without having to differentiate color hues and intensities.

### BRIEF DESCRIPTION OF THE DRAWINGS

The accompanying drawings, which are incorporated in and form a part of the specification, illustrate embodiments of the apparatus of the present invention and, together with the description, serve to explain the principles of the invention. In the drawings:

FIG. 1A is a schematic representation of a top view of an embodiment of the elongated substrate of the capillarity-based analytic device hereof, illustrating a liquid-confining path formed therethrough onto which colorimetric reagents that are effective for reacting with a specific analyte are deposited, and a liquid well formed at one end thereof; FIG. 1B is a schematic representation of a top view of the assembled device, showing a scale imprinted on either the surface of the assembled device or on the substrate, and an orifice in the top liquid impermeable surface permitting fluid access to the liquid well of the substrate; FIG. 1C is a schematic representation of a side view of the assembled apparatus, illustrating the substrate shown in FIG. 1A having a liquid impervious coating on both sides thereof; and FIG. 1D is a perspective view of the assembled device illustrated in FIG. 1B, showing an expanded view of the orifice thereof.

FIG. 2A is a schematic representation of a top view of another embodiment of the elongated substrate of the capillarity-based analytic device hereof, illustrating a liquid-confining path formed by the substrate itself into which colorimetric reagents effective for reacting with a specific analyte are deposited, and a liquid well formed on one end thereof; FIG. 2B is a schematic representation of a top view of the assembled device, showing a scale imprinted on either the surface of the assembled device or on the substrate, and an orifice in the top surface permitting fluid access to the liquid well of the substrate; FIG. 2C is a schematic representation of a side view of the assembled apparatus, illustrating the substrate shown in FIG. 2A enclosed in a liquid impervious coating on both sides thereof; and FIG. 2D is a perspective view of the assembled device illustrated in FIG. 2B, showing an expanded view of the orifice thereof.

FIG. 3A is a schematic representation of a top view of an embodiment of the elongated substrate of the capillarity-based analytic device hereof, illustrating a liquid-confining path formed thereon similar to that shown in FIG. 1A hereof, except that the liquid confining path is not linear, but provides a more circuitous route along the substrate in situations where reaction kinetics are slow, and onto which colorimetric reagents effective for reacting with a specific analyte are deposited, and a liquid well formed at one end thereof; FIG. 3B is a schematic representation of a top view of the assembled device, showing a scale imprinted on either the surface of the assembled device or on the substrate, and an orifice in the top surface permitting fluid access to the liquid well of the substrate; FIG. 3C is a schematic representation of a side view of the assembled apparatus, illustrating the substrate shown in FIG. 3A having a liquid impervious coating on both sides thereof; and FIG. 3D is a perspective view of the assembled device illustrated in FIG. 3B, showing an expanded view of the orifice thereof.

FIG. 4 illustrates fabrication and assembly of the embodiment of the device shown in FIGS. 1A-1D, hereof.

FIG. 5 illustrates fabrication and assembly of the embodiment of the device shown in FIGS. 2A-2D, hereof.

FIG. 6 illustrates fabrication and assembly of the embodiment of the device shown in FIGS. 3A-3D, hereof.

FIG. 7A is a graph of the distance in millimeters of color development in the apparatus illustrated in FIG. 1 hereof, as a Log function of a known quantity of analyte in nmols for a glucose analysis system, FIG. 7B as a Log function of a known quantity of analyte in nmols for a glutathione analysis system, and FIG. 7C as a function of a known quantity of analyte in nmols for a nickel analysis system, all within the linear range of the reaction, the error bars representing one standard deviation, and the diagrams of the complete reaction are included for each calibration data point.

### DETAILED DESCRIPTION OF THE INVENTION

Embodiments of the present invention include a simple apparatus for quantitative, capillarity-based analyses having broad chemical applicability (See, “Simple, Distance-Based Detection for Paper Analytical Devices,” by David M. Cate et al., Lab on a Chip 13 (12): 2397-2404 (25 April 2013) doi:10.1039/C3LC50072A which is hereby incorporated by reference herein for all that it discloses and teaches.). Hydrophobic materials may be printed onto the paper for defining flow circuits or paths that restrict liquid flow by capillary action to defined regions. At least one colorimetric reagent effective for reacting with a specific analyte is deposited along a capillary flow path generated in the capillarity-based device. Upon placing the liquid containing the analyte on one end of the circuit, the liquid moves along the path by capillary action, whereby as the flowing analyte reacts with reagent, color develops along the flow path until all of the analyte is consumed. Analyte quantification is achieved by measuring the length of the colored portion along the flow path, using a direct-reading measurement scale formed alongside or on the flow path, thus eliminating the need to differentiate color hues and intensities by the user as is typical with existing PADs. Assays based on color length were developed that use enzymatic action, metal complexation, and nanoparticle aggregation. Each assay provided quantitative detection of different analytes within specific biological and environmental matrices of interest.

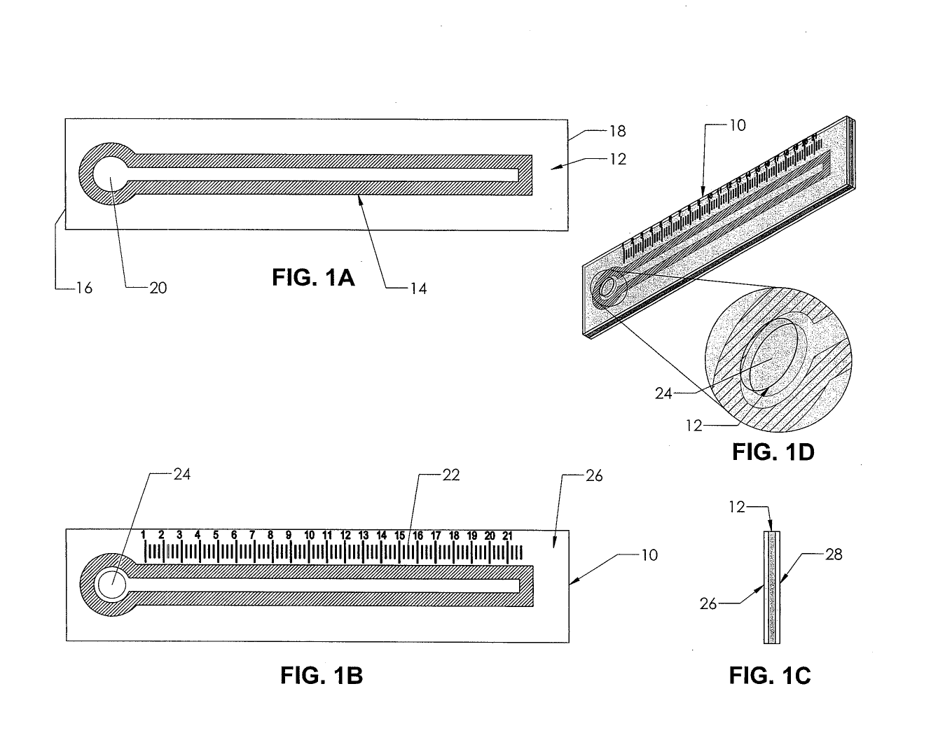

Reference will now be made in detail to the present embodiments of the invention, examples of which are illustrated in the accompanying drawings. In the FIGURES, similar structure will be identified using identical reference characters. It will be understood that the FIGURES are for the purpose of describing particular embodiments of the invention and are not intended to limit the invention thereto. Turning now to FIG. 1, an embodiment of the capillarity-based analytic device, 10, of the present invention is illustrated, where FIG. 1A is a schematic representation of a top view of an embodiment of elongated substrate, 12, illustrating a liquid-confining path, 14, formed thereon having a first end, 16, and a second end, 18, into which colorimetric reagents effective for reacting with a specific analyte are deposited, and fluid well, 20, formed near first end, 16, thereof. A wax ink may be designed and printed onto substrate 12, using graphics software, and subsequently heated to generate a two-dimensional liquid-confining channel, the top and bottom confinement being generated using liquid-impervious sheeting, as will be described in more detail hereinbelow. The substrate used for the analyses set forth in the EXAMPLES hereinbelow was standard cellulosic filter paper. However, any porous hydrophilic material that can be patterned or cut into the desired shape may be used for such assays. Other examples include, glass, nitrocellulose, silk, and cotton. For non-aqueous, non-polar systems, hydrophobic substrates such as nylon, Polytetrafluoroethylene (PTFE), Polyvinylidene fluoride (PVDF), or other halogenated polymers capable of providing sufficient chemical resistance and effective for wicking non-polar organic solvents, may be used. Colorimetric detection reagents were deposited along the flow channel by spray application or by use of a pipette, as examples. For spray application, a nebulizer is used to deposit reagent droplets uniformly along the channel. This process is rapid, but inefficient, since significant amounts of reagent are deposited onto the surrounding paper; that is, outside of the flow circuit. Although these reagents do not affect the assay results because they are separated from the flow channel by the wax barrier, they are wasteful. Alternatively, a pipette was used to deposit the reagents onto the paper in minute (approximately 0.5 μL, as an example) increments, which provides more efficient use of reagents. Once the deposited reagents are dry, device 10 is ready for use.

FIG. 1B is a schematic representation of a top view of assembled device 10 showing direct-reading measuring scale, 22, imprinted either on the surface of assembled device 10 or on substrate 12, and orifice, 24, in top liquid impervious surface, 26, permitting access to liquid well 20 of substrate 12, for sample addition. Substrate 12 below orifice 24 may be retained for holding reagents for sample pre-treatment, or removed to facilitate sample transfer into the detection zone. As stated hereinabove, a liquid sample is introduced into the sample reservoir and then carried by capillary action along the flow channel. As the analyte reacts with its reagent, a colored product develops. Once all of the analyte has reacted, the color development stops (even though the solution continues to flow along the channel), the developed colored product remaining where it was generated. Analyte quantification is then performed by measuring the length of the colored region on the flow channel using the direct-reading measurement scale. Samples were introduced into the sample well using a syringe. Other methods of sample introduction include: (a) dipping the well portion of the device directly into a liquid solution containing the analyte; (b) using inertial impaction to deposit airborne particulate matter into the well and solubilizing the particulate matter using a suitable liquid for dissolving and carrying the dissolved material into the channel; and (c) flowing gas (or liquid) through the sample addition well orthogonal to the capillarity flow path, assuming there was no backing/laminate. Some portion of the analyte in the reservoir could then be trapped or sequestered, or the orthogonal flow is allowed to migrate into the analysis channel, thereby introducing analyte into the channel.

FIG. 1C is a schematic representation of a side view of the assembled apparatus, illustrating substrate 12 having liquid impervious layers, 26, and, 28, on both sides thereof. FIG. 1D is a perspective view of the assembled device 10 illustrated in FIG. 1B, showing an expanded view of orifice 24 thereof.

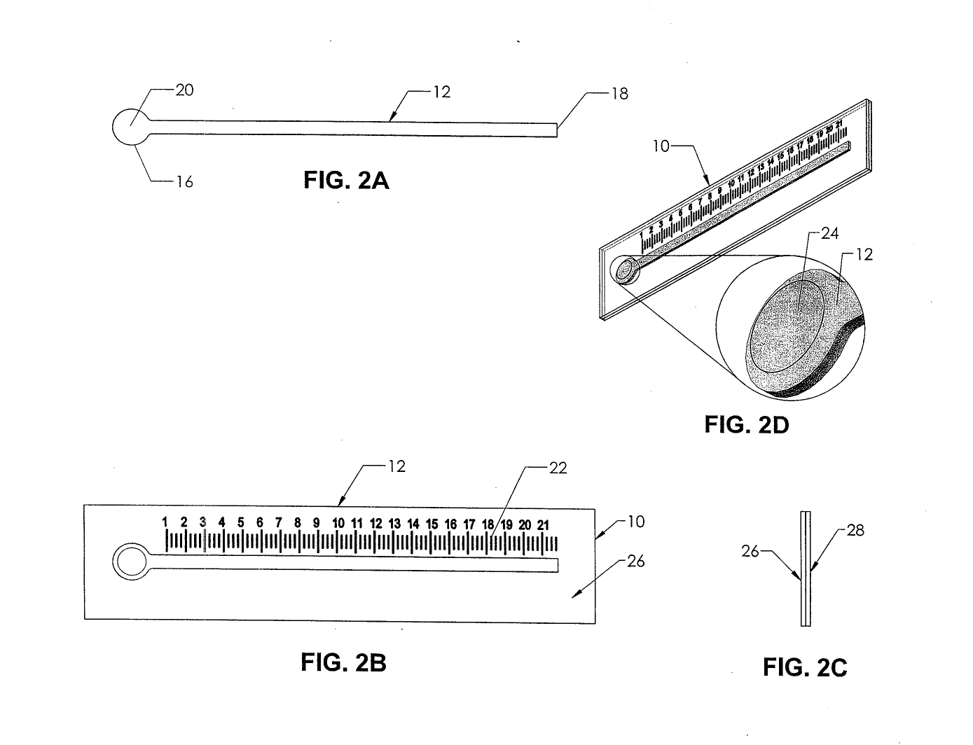

FIG. 2A is a schematic representation of a top view of a second embodiment of elongated substrate 12 of the capillarity-based analytic device 10 hereof. In this embodiment, the liquid-confining path is formed by substrate 12, itself, when sandwiched between two liquid-impervious sheets, there being no requirement to use a wax ink as described hereinabove. Colorimetric reagents effective for reacting with a specific analyte are deposited onto substrate 12 before it is covered. Liquid well 24 is formed near end 20 thereof. FIG. 2B is a schematic representation of a top view of assembled device 10, showing direct-reading measuring scale 22 imprinted on liquid-impervious surface 26 of assembled device 10, and orifice 24 in top, liquid-impervious surface 26, whereby liquid is permitted access to liquid well 20 of substrate 12. FIG. 2C is a schematic representation of a side view of the assembled apparatus, illustrating substrate 12 shown in FIG. 2A hereof enclosed by liquid impervious layers 26 and 28. FIG. 2D is a perspective view of assembled device 10 illustrated in FIG. 2B, hereof showing an expanded view of orifice 24 thereof.

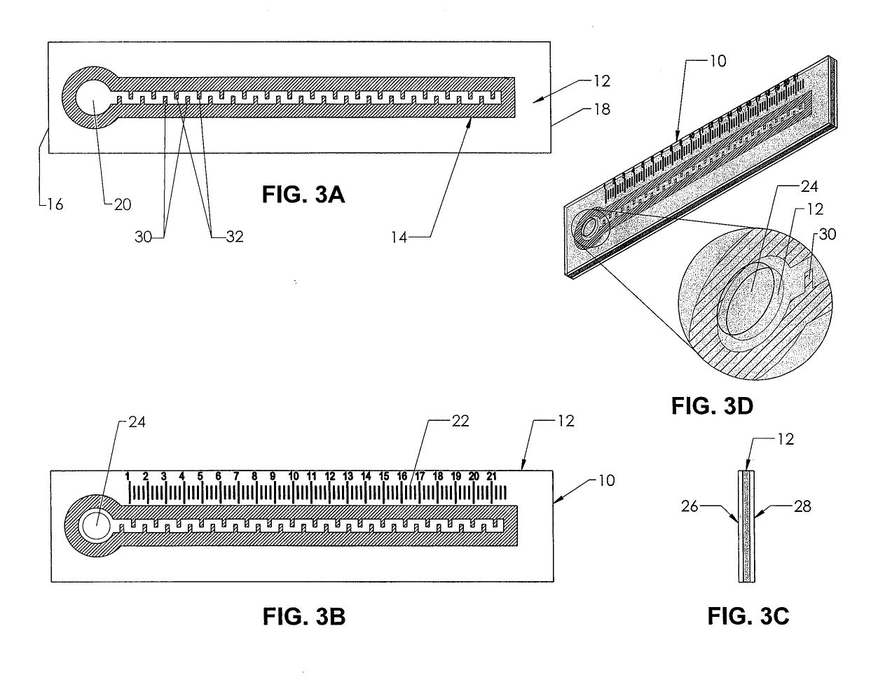

FIG. 3A is a schematic representation of a top view of an embodiment of elongated substrate 12 of capillarity-based analytic device 10 hereof, illustrating liquid-confining path 14 formed therethrough in a similar manner to that described for FIG. 1A hereof, except that the liquid confining path is not linear, but provides a more circuitous route along the substrate for situations where the reaction kinetics are slow. As an example, wax baffles, 30, and, 32, may be printed on substrate 12, and used to divert liquid flow in a nonlinear manner. Colorimetric reagents effective for reacting with the analyte are again deposited, and liquid well 20 is formed near end 16 thereof. FIG. 3B is a schematic representation of a top view of assembled device 10, showing scale 22 imprinted either on liquid impermeable surface 26 of device 10, or on substrate 12, and orifice 24 in top liquid impermeable surface 26, thereby permitting fluid access to liquid well 20 of substrate 12. FIG. 3C is a schematic representation of a side view of the assembled apparatus, illustrating substrate 12 having liquid impervious layers 28 and 30 on either side thereof. FIG. 3D is a perspective view of assembled device 10 illustrated in FIG. 3B, showing an expanded view of orifice 24 thereof.

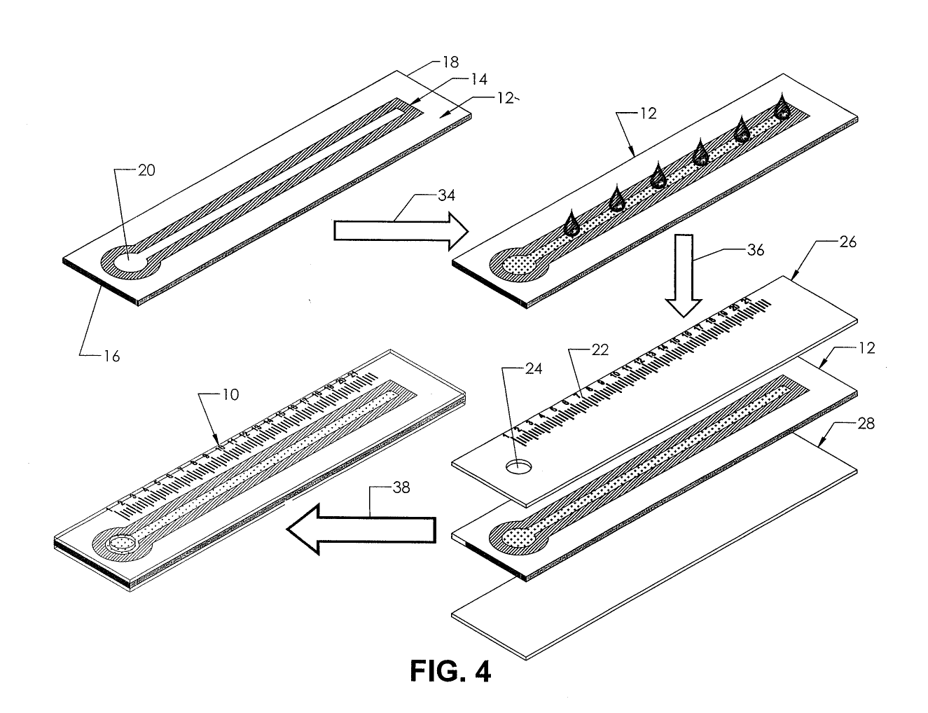

FIG. 4 illustrates fabrication and assembly of the embodiment of the device shown in FIGS. 1A-1D, hereof. After a wax ink is printed onto substrate 12, and subsequently heated to generate a two-dimensional liquid-confining channel 14, step 34, shows the deposition of colorimetric reagents effective for reacting with a specific analyte, by spray application or by pipetting, as examples. The reagents are then allowed to dry. Step 36 illustrates the placement of transparent, liquid-impervious sheet 26 having measuring scale 22 printed thereon and having hole or orifice 24 therein to permit liquid access to liquid well, 20, formed near first end, 16, thereof, onto substrate 12, and the placement of second liquid impervious sheet 28, which may not be transparent, onto the bottom of substrate 12. Step 38 seals sheets 26 and 28 to substrate 12, using a thermal laminating process, as an example, as is known in the art, completing measuring apparatus 10. The sealing of sheets 28 and 30 to substrate 12 completes the formation of liquid confinement channel. Clearly, other methods for creating a liquid impermeable barrier on substrate 12 are envisioned, one being a coating process.

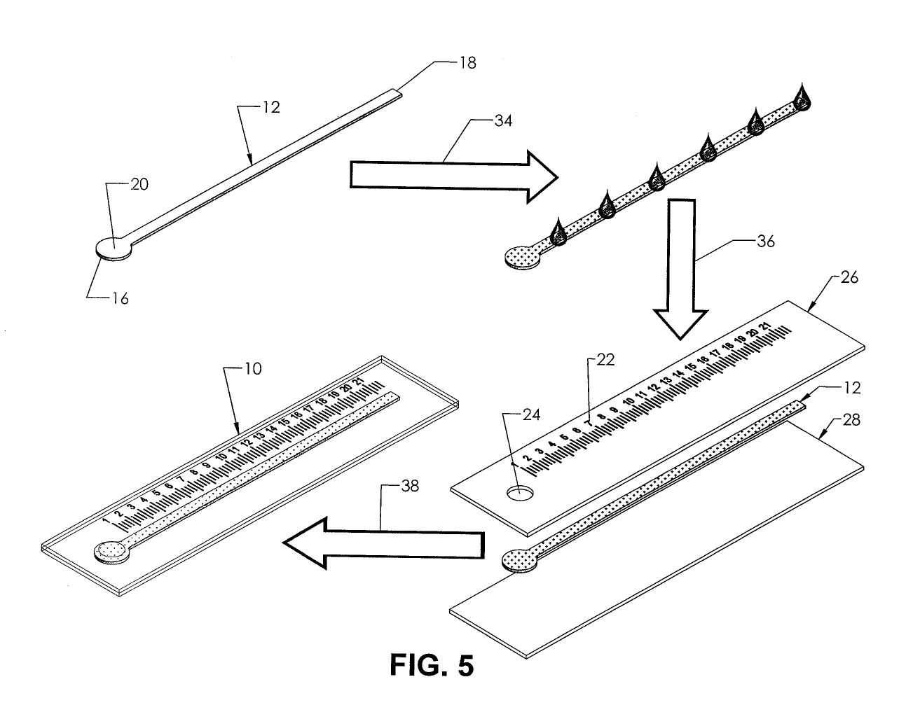

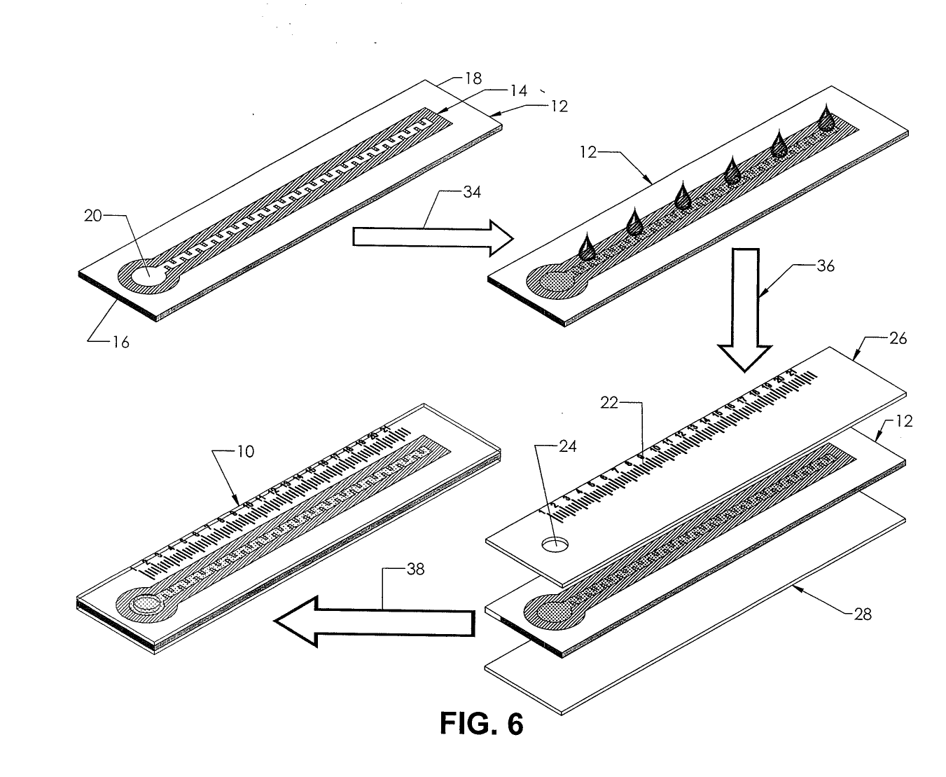

FIG. 5 illustrates fabrication and assembly of the embodiment of the device shown in FIGS. 2A-2D, hereof, and FIG. 6 illustrates fabrication and assembly of the embodiment of the device shown in FIGS. 3A-3D, hereof, by similar process steps to those shown in FIG. 3.

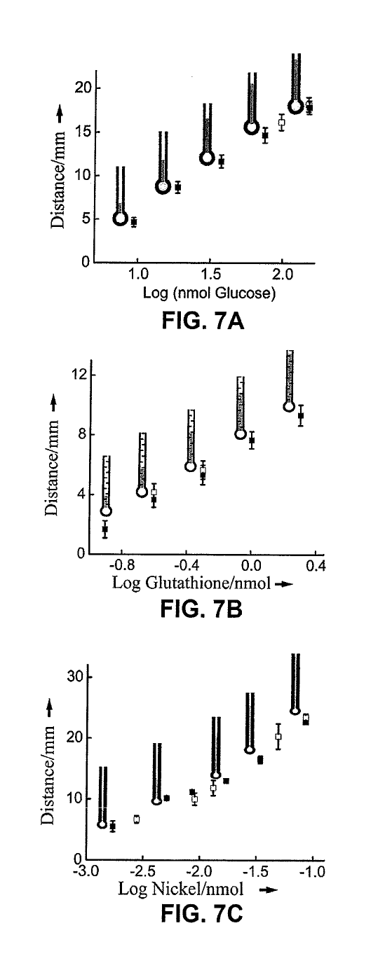

FIG. 7A is a graph of the distance in millimeters of color development in the apparatus illustrated in FIG. 1 hereof, as a Log function of a known quantity of analyte in nmols for a glucose analysis system, FIG. 7B as a Log function of a known quantity of analyte in nmols for a glutathione analysis system, and FIG. 7C as a function of a known quantity of analyte in nmols for a nickel analysis system, all within the linear range of the reaction, the error bars representing one standard deviation, and the diagrams of the complete reaction are included for each calibration data point. Glucose was detected using glucose oxidase, 3,3′-diaminobenzidine (DAB) and peroxidase, where the glucose oxidase produces hydrogen peroxide that further reacts with DAB in the presence of peroxidase to form a brown, insoluble product (polyDAB). Like DMG, DAB is colorless, but forms a highly colored and easily visualized product in the presence of the analyte. Glutathione (GSH) was detected using a silver nanoparticle (AgNP) aggregation assay, where the AgNPs aggregate in the presence of GSH to form a reddish-brown product that is distinguished from the orange color of the AgNPs in the absence of glutathione. Nickel, as Ni²⁺, was detected using dimethylglyoxime (DMG) as an example assay for heavy metals, where DMG is placed in the channel and reacts with Ni²⁺to form a pink product. Solutions containing Ni²⁺are colorless in the absence of DMG. These reactions will be described in more detail in the EXAMPLES set forth hereinbelow.

Capillarity-based analytical devices have great potential for application at the point-of-need. The quantitative analytical device of embodiments of the present invention is minimally instrumented for device portability, and is highly cost effective; excluding fabrication equipment, a single assay costs approximately $0.04. Since analyte quantification is immediate and can be performed on-site, processing time is significantly reduced when compared to other centralized measurement techniques, which often sacrifice processing speed for detection sensitivity. Like most PAD technologies, however, embodiments of the present invention sacrifice dynamic range for cost, speed, and ease of use. This limitation on reaction stoichiometries can be accommodated in part by tuning the capillarity-based analytical devices hereof to detect different analyte concentration ranges by adjusting reagent concentrations in the flow channel.

Having generally described the invention, the following EXAMPLES provide greater detail. In what follows, cellulosic filter paper was used as the substrate.

### EXAMPLE 1

### Glucose Detection:

Human control serum samples (levels I and II) for both GSH and glucose were obtained from commercial sources. Levels of analytes were provided by the suppliers. Before analysis, unwanted protein was removed from samples using a filter (10 kDa MWCO) and centrifuging for 20 min. at 10,000 rpm for glucose and 10 min. for GSH. In addition, a solution of 5% 5-sulfosaliccylic acid was added prior to centrifugation for GSH.

The capillarity-based paper-based assay for glucose detection consisted of a wax-printed circular reservoir (5 mm diameter) for glucose oxidase (GOD) and peroxidase Type I (HRP) enzyme modification, and a straight channel (2 mm×40 mm) for measuring glucose reaction with peroxidase and DAB. Aliquots (˜0.5 μL) of 600 U/mL glucose oxidase and 500 U/mL HRP were spotted on the sample reservoir and ˜0.5 μL of DAB was pipetted onto the straight channel every five millimeters to account for reagent spreading along the channel length. For each assay, ˜20 μL of the standard or sample solution was added to the sample reservoir. The length of the colored range was found to be proportional to the amount of glucose added over the range of ˜7 nmol to ˜200 nmol. Method variability was relatively low as seen by the small error bars (representing standard deviations of repeat measures) around each datum as illustrated in FIG. 7A. Commercially-available control serum samples known to contain either normal or abnormal glucose levels were also analyzed. Glucose concentrations within the control serum samples are shown in FIG. 7A as open squares; their alignment with the calibration curve shows the ability of this method to measure glucose accurately and precisely in a relatively complex sample matrix.

### EXAMPLE 2

### Glutathione Detection:

The paper assay for glutathione detection consisted of a circular reservoir for sample addition (6 mm diameter) and a baffled flow channel (3 mm×60 mm) divided into 14 equal sections (0.3 mm×2 mm). Flow baffles were used to decrease the capillary flow velocity along the channel, thereby maximizing reaction time between glutathione and the AgNPs. The AgNP solution (˜0.5 μL) was spotted onto each of the 14 sections along the channel. For each assay, ˜20 μL of sample solution was added to the sample reservoir. Complete sample analysis took approximately 10 min. Assay selectivity was investigated by addition of ˜20 μL of standard thiol solution (˜0.5 nmol), which did not form a colored reaction product along the paper channel.

The spotted detection reagent, AgNP (˜11 nm diameter) turned a dark orange color. The nanoparticles aggregate in the presence of glutathione, which causes a color change from orange to deep red on the paper substrate. A color change from orange to light orange was observed when buffer was added, but was easily distinguished from the dark red of the glutathione-specific product. Detection of glutathione was log-linear for the concentration range tested (˜0.12 nmol to ˜2.0 nmol). The assay selectivity against other thiols (cysteine and homocysteine) and disulfides (cysteine, homocystine, and glutathione disulfide) was also determined. Cysteine and homocysteine were found to cause similar color changes, but the length of color development was much less than for glutathione. None of the disulfides tested caused any color change. The ability to measure glutathione spiked in serum samples (open squares on FIG. 7B) was determined. The measured distances in serum (˜4.2 and ˜5.7 mm) agree well with those of the standard solutions (˜3.7 and ˜5.3 mm) for glutathione concentrations ˜0.25 and ˜0.5 nmol respectively.

### EXAMPLE 3

### Nickel Detection:

A nebulizer was used to saturate the paper surface with DMG (˜50 mM). The deposited reagents were then air dried. The paper was uniformly coated with ammonium hydroxide (pH 9.5), because the rate and extent of Ni²⁺-DMG complexation are pH dependent, with the fastest rate occurring at a pH of 9. To prevent user contamination and excess solvent evaporation, the filter paper was passed through a desktop laminator at 300° F. twice on each side. Laminating the paper also provided better mechanical stability for assay handling. A ˜6.4 mm (ID) hole was punched through the sample reservoir and masking tape was applied to one side to prevent sample loss from leakage during use. For analysis, ˜20 μL of a Ni standard solution (1000 ppm) was deposited onto the sample reservoir. The Ni-DMG complex is reddish pink, precipitates upon formation, and was readily distinguished from the clear sample solution. Color development is rapid and total sample analysis was performed in less than ten minutes. The reaction distance was measured using the naked eye and verified using a desktop scanner. It was found that as the amount of DMG increases, the sensitivity of the assay increases. The assay detection limits are sufficiently low that nmol levels of Ni²⁺can be detected in the presence of other transition and heavy metals. To measure Ni concentration, the incineration ash was first dissolved in acid and then treated to complex interfering metals. Various dilutions of the resulting solution were analyzed, and the results shown as open squares in FIG. 7C.

An incineration ash sample was purchased for assay validation. Incineration ash along with ˜1 mL concentrated nitric acid was heated in a 20 mL scintillation vial for five min. at ˜250° C. on a hotplate until complete acid evaporation. An ˜262 μL solution containing deionized water (˜250 μL), sodium fluoride, acetic acid (2:1:1 v/v %), and ˜12 μL sodium hydroxide (12 M) was added to the vial. After homogenous mixing with a pipette for several seconds, the solution was centrifuged for 10 min. at 14,000 RPM. For each assay, ˜20 μL of the supernatant was added to the sample reservoir. Good agreement was obtained between measured and known Ni concentrations.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. Apparatus for capillarity-based, quantitative analysis of an analyte dissolved in a liquid, comprising:
an elongated substrate effective for wicking said liquid;
means for confining said liquid to a defined elongated path having a first end along said substrate, forming thereby a capillary flow path into which at least one colorimetric reagent effective for reacting with said analyte, is deposited;
means for introducing a chosen portion of said liquid into capillary flow path of the elongated path at a location in the region of the first end thereof;
whereby said liquid moves along the capillary flow path of the elongated path away from the first end by capillary action and, as the flowing analyte reacts with said at least one reagent, color develops along the flow path to a distance from the location of introduction thereof where all of said analyte is reacted; and
means for measuring the distance between the region of the first end of the elongated path and the location of where all of said analyte is reacted.

2. The apparatus of claim 1, wherein said liquid comprises water.

3. The apparatus of claim 2, wherein said means for confining said liquid to the defined elongated path comprises a hydrophobic material applied to said substrate such that an elongated path is defined.

4. The apparatus of claim 3, wherein said hydrophobic material comprises at least one wax.

5. The apparatus of claim 4, wherein said at least one wax is printed onto said substrate.

6. The apparatus of claim 4, wherein said at least one wax is melted into said substrate.

7. The apparatus of claim 1, wherein said liquid comprises a non-polar organic solvent.

8. The apparatus of claim 1, wherein the elongated path extends a chosen length along said substrate, defining an axis along said substrate.

9. The apparatus of claim 8, wherein the elongated path comprises a linear path.

10. The apparatus of claim 8, wherein the elongated path crosses the axis a plurality of times along said chosen length.

11. The apparatus of claim 1, wherein said means for introducing a chosen portion of said liquid into the elongated path comprises a syringe.

12. The apparatus of claim 2, wherein said substrate comprises porous cellulose.

13. The apparatus of claim 12, wherein said substrate comprises filter paper.

14. The apparatus of claim 2, wherein said substrate is chosen from glass, nitrocellulose, silk, and cotton.

15. The apparatus of claim 7, wherein said substrate comprises a hydrophobic substrate.

16. The apparatus of claim 15, wherein said substrate is chosen from nylon, Polytetrafluoroethylene, and Polyvinylidene fluoride.

17. The apparatus of claim 1, wherein said means for measuring the distance between the region of the first end of the elongated path and the location of termination of the color comprises a measurement scale printed on said substrate.

18. The apparatus of claim 1, wherein said means for measuring the distance between the region of the first end of the elongated path and the location of termination of the color comprises a ruler.

19. The apparatus of claim 3, wherein said substrate has a top surface and a bottom surface, and said means for confining said liquid to a defined elongated path having a first end along said substrate, further comprises a first transparent liquid impervious layer in contact with the top surface of said substrate, and a second liquid impervious layer in contact with the bottom surface of said substrate, said first layer and said second layer forming a seal around said substrate, and wherein said first layer has an orifice therein in the region of the first end of the elongated path opening to said substrate.

20. The apparatus of claim 19, wherein said means for measuring the distance between the region of the first end of the elongated path and the location of termination of the color comprises a measurement scale printed on said substrate.

21. The apparatus of claim 19, wherein said means for measuring the distance between the region of the first end of the elongated path and the location of termination of the color comprises a direct-reading measurement scale printed on said first liquid impervious layer.

22. Apparatus for capillarity-based, quantitative analysis of an analyte dissolved in a liquid, comprising:
an elongated substrate effective for wicking said liquid;
a liquid repelling material applied to said substrate such that an elongated path is defined for confining said liquid to a defined elongated path having a first end along said substrate, forming thereby a capillary flow path into which at least one colorimetric reagent effective for reacting with said analyte, is deposited;
a syringe for introducing a chosen portion of said liquid into capillary flow path of the elongated path at a location in the region of the first end thereof;
whereby said liquid moves along the capillary flow path of the elongated path away from the first end by capillary action and, as the flowing analyte reacts with said at least one reagent, color develops along the flow path to a distance from the location of introduction thereof where all of said analyte is reacted; and
a measurement scale printed on said substrate for measuring the distance between the region of the first end of the elongated path and the location of where all of said analyte is reacted.

23. The apparatus of claim 22, wherein said substrate has a top surface and a bottom surface, said apparatus further comprising a first transparent liquid impervious layer in contact with the top surface of said substrate, and a second liquid impervious layer in contact with the bottom surface of said substrate, said first layer and said second layer forming a seal around said substrate, and wherein said first layer has an orifice therein in the region of the first end of the elongated path opening to said substrate.

24. Apparatus for capillarity-based, quantitative analysis of an analyte dissolved in a liquid, comprising:
an elongated substrate having a top surface and a bottom surface, effective for wicking said liquid;
a liquid repelling material applied to said substrate such that an elongated path is defined for confining said liquid to a defined elongated path having a first end along said substrate, forming thereby a capillary flow path into which at least one colorimetric reagent effective for reacting with said analyte, is deposited;
a syringe for introducing a chosen portion of said liquid into capillary flow path of the elongated path at a location in the region of the first end thereof;
whereby said liquid moves along the capillary flow path of the elongated path away from the first end by capillary action and, as the flowing analyte reacts with said at least one reagent, color develops along the flow path to a distance from the location of introduction thereof where all of said analyte is reacted;
a first transparent liquid impervious layer in contact with the top surface of said substrate, and a second liquid impervious layer in contact with the bottom surface of said substrate, said first layer and said second layer forming a seal around said substrate, and wherein said first layer has an orifice therein in the region of the first end of the elongated path opening to said substrate; and
a direct-reading measurement scale printed on said first liquid impervious layer for measuring the distance between the region of the first end of the elongated path and the location where all of said analyte is reacted.
